(19) United States
(12) Patent Application Publication
(10) Pub. No.: US 2014/0251925 A1
(43) Pub. Date: Sep. 11, 2014

(54) Bio-fluid treatment device and method

(71) Applicant: AVIcure Bioscience, LLC, Bethlehem, PA (US)

(72) Inventor: Thomas R. Petrie, Hamburg, NJ (US)

(73) Assignee: AVIcure Bioscience, LLC, Bethlehem, PA (US)

(21) Appl. No.: 13/815,584

(22) Filed: Mar. 11, 2013

(51) Int. Cl. A61M 1/36

(52) U.S. Cl. CPC A61M 1/3681
USPC 210/748.11; 250/432 R

(57) ABSTRACT

A treatment device and method for treatment of a bio-fluid are disclosed. The device includes a treatment chamber configured to receive bio-fluid to be treated, a light treatment element disposed around at least a portion of the treatment chamber, and a piston mechanism disposed inside the treatment chamber for advancing the bio-fluid inside the treatment chamber toward a constricted location corresponding to the at least the portion of the treatment chamber around which the treatment element is being disposed. Upon passing bio-fluid through the location, the treatment element applies a treatment to the passing bio-fluid.

### CROSS-REFERENCE TO RELATED APPLICATIONS

The present application relates to U.S. patent application Ser. No. 11/285,959 to Petrie, filed Nov. 22, 2005, and entitled “Blood Irradiation System, Associated Devices and Methods for Irradiating Blood”, now U.S. Pat. No. 7,547,391 B2 and U.S. and U.S. Pat. No. 6,312,593 B1 to Petrie, filed Apr. 23, 1999, issued Nov. 6, 2001, and entitled “Ultraviolet Blood Irradiation Chamber”. The present application incorporates the disclosures of the above applications and patent herein by reference in their entireties.

### BACKGROUND OF THE INVENTION

1. Field of the Invention

Embodiments of the present invention are directed to devices, systems and methods for treating bio-fluids (e.g., blood and blood products, such as platelets, red cells and plasma) with ultraviolet light, and corresponding related components, systems and methods.

2. Background

It has long been recognized and understood that specific wavelengths of ultraviolet radiation have the ability to destroy certain biological and chemical structures. While the sun and most active celestial bodies normally emit all types of UV radiation, portions of the earth's atmosphere prevent its destructive form of energy from reaching the surface.

During the last century, scientists and medical practitioners experimented with the use of UV radiation in the treatment of diseases. One such experiment in the late 1930's involved the development of a rudimentary device designed to expose human blood to a UV lamp, in an effort to kill virus and bacteria. This particular device, while sometimes medically successful with respect to the patients being treated, was an electrical and mechanical failure due to several factors. First and foremost, the UV lamp was difficult to operate; just to get the lamp to strike was a major handling problem. There were numerous interactive controls that required constant re-adjustment to keep the device operating properly. In addition, the lamp had only a short lifespan before it either failed to strike, or produce the necessary therapeutic wavelength of UV. There was also an ongoing general maintenance issue with a water cooling process and a belt drive sequence of included mechanics. In addition, the control of the flow rate of the blood through the system also required constant adjustment and monitoring by a trained operator. Because of the design of the device, blood collection was also difficult. Specifically, gravity was used to draw and collect the blood into an open beaker. The beaker was than moved to a position above the device and allowed to drain through the pump and exposure chamber.

Although positive therapeutic treatments sometimes resulted when all system components were operating properly, such conditions did not occur often. Moreover, if a mechanical, electrical or lamp problem developed during the course of a clinical procedure, the system provided no visual or audible indications to notify the operator or an automatic fail-safe termination of operation.

### SUMMARY OF THE INVENTION

In some embodiments, the present invention is directed to a device that exposes bio-fluids, especially whole blood or blood products, to an Ultraviolet (UV) irradiation energy source for sterilization or chemical reaction during the action of infusion, collection, removal or bulk processing, e.g. in a blood banks, clinics and hospital environments. Some target end products that are sterilized include concentrated red cells and plasma. In some embodiments, the device includes a controlled thin film displacement reactor that enables direct and reflective UV exposure for sterilization or reaction. The reactor and UV source are located in-line either with the intra-venous blood processing lines, collection bags/containers or bulk blood containers. In some embodiments, the present invention relates to a treatment device for treatment of a bio-fluid. The device includes a treatment chamber configured to receive bio-fluid to be treated, a light treatment element disposed around at least a portion of the treatment chamber, and a piston mechanism disposed inside the treatment chamber for advancing the bio-fluid inside the treatment chamber toward a constricted location corresponding to the at least the portion of the treatment chamber around which the treatment element is being disposed. The constricted location has a passage width of about 1 millimeter to about 5 millimeters to permit optimal light penetration through said bio-fluid (as used herein “width” should be taken to mean the narrowest inside cross section through which the bio-fluid flows within the chamber's constricted location-hence, it could be an inside diameter, or a narrowest cross section of any shape utilized for the constricted location. In some preferred embodiments, the preferred passage width is about 2 to about 4 millimeters. Upon passing of the bio-fluid through the constricted location, the treatment element applies a treatment to the passing bio-fluid.

In some embodiments, the bio-fluid is blood or one or more blood products, and the treatment element is an ultraviolet (“UV”) lamp, where the treatment is application of UV radiation.

In some embodiments, the treatment device includes cooling mechanisms disposed along the treatment chamber and wherein the treatment element is configured to be disposed around the cooling mechanism and the treatment chamber. Each the cooling mechanism includes a cooling jacket containing water, where the water is distilled and air-free. Further, the cooling mechanisms are configured to reduce temperature of bio-fluid inside the treatment chamber during the treatment.

In some embodiments, the piston mechanism is configured to be coupled to a motor. The motor is configured to simultaneously translate and rotate and vertically pulse the piston mechanism inside the treatment chamber during the treatment, thereby causing the bio-fluid to spread along interior walls of the treatment chamber. Further, the treatment chamber can be further coupled to an untreated bio-fluid container for supplying bio-fluid to the treatment chamber using a pumping mechanism. Also, upon receiving bio-fluid from the untreated bio-fluid container, the piston mechanism is configured to advance the received bio-fluid inside the treatment chamber for treatment. Upon treating the bio-fluid inside the treatment chamber using the treatment element, the treated bio-fluid is collected inside a collection container coupled to the treatment chamber.

In some embodiments, the present invention relates to a method for treating a bio-fluid using a treatment device for treatment of a bio-fluid. The treatment device includes a treatment chamber configured to receive bio-fluid to be treated, a light treatment element disposed around at least a portion of the treatment chamber, a piston mechanism disposed inside the treatment chamber for advancing the bio-fluid inside the treatment chamber toward a constricted location corresponding to at least a portion of the treatment chamber around which the treatment element is being disposed. The constricted location has a passage width of about 1 millimeter to about 5 millimeters to permit optimal light penetration through said bio-fluid (as used herein “width” should be taken to mean the narrowest inside cross section through which the bio-fluid flows within the chamber's constricted location—hence, it could be an inside diameter, or a narrowest cross section of any shape utilized for the constricted location. In some preferred embodiments, the preferred passage width is about 2 to about 4 millimeters. Upon passing of the bio-fluid through the constricted location, the treatment element applies a treatment to the passing bio-fluid. The method includes receiving bio-fluid to be treated, using the piston mechanism, advancing the bio-fluid inside the treatment chamber, treating the bio-fluid inside the treatment chamber, and, collecting the treated bio-fluid in a collection container coupled to the treatment chamber. In some embodiments, the bio-fluid is blood and the treatment element is an ultraviolet (“UV”) lamp, where the treatment is application of UV radiation. The method can include cooling of the bio-fluid inside the treatment chamber during the treating step.

These and other embodiments, features, advantages and objects of the invention will become even more apparent with reference to the following detailed description and attached drawings, a brief description of which is set out below.

### BRIEF DESCRIPTION OF THE DRAWINGS

FIG. 1 illustrates an exemplary blood sterilization system, according to some embodiments of the present invention.

FIG. 2 is a perspective view of a blood sterilization device, according to some embodiments of the present invention.

FIG. 3 is an exploded perspective view of the blood sterilization device shown in

FIG. 2.

FIG. 4 is another perspective view of the blood sterilization device shown in FIG. 2.

FIG. 5 is another exploded perspective view of the blood sterilization device shown in FIG. 2.

FIG. 6 is yet another exploded perspective view of the blood sterilization device shown in FIG. 2.

FIG. 7 is a cross-sectional view of the blood sterilization device shown in FIG. 2.

FIG. 8 is yet another exploded perspective view of the blood sterilization device shown in FIG. 2.

FIG. 9 is a cross-sectional view of several components of the blood sterilization device shown in FIG. 2 and illustrates blood flow through the blood sterilization device.

FIG. 10 is yet another cross-sectional view of the blood sterilization device shown in FIG. 2 showing a lamp and an exposure window.

FIG. 11 illustrates an exemplary blood sterilization device, according to some embodiments of the present invention.

FIG. 12 illustrates an exemplary chamber of a blood sterilization device, according to some embodiments of the present invention.

FIG. 13 illustrates yet another exemplary blood sterilization device, according to some embodiments of the present invention.

### DETAILED DESCRIPTION OF THE INVENTION

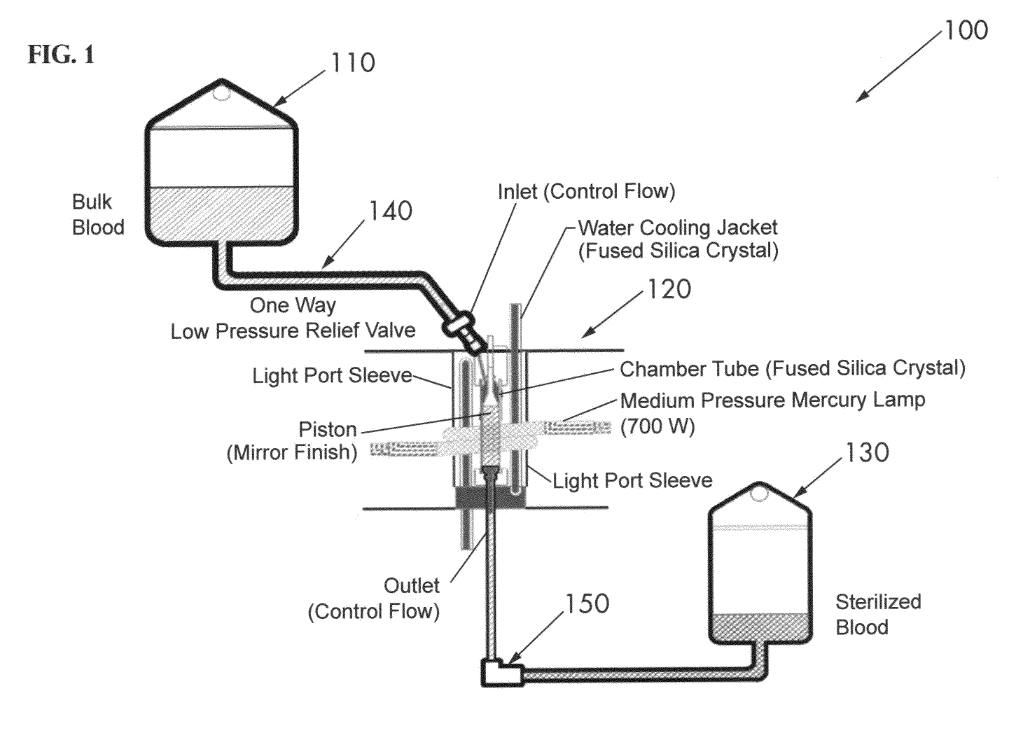

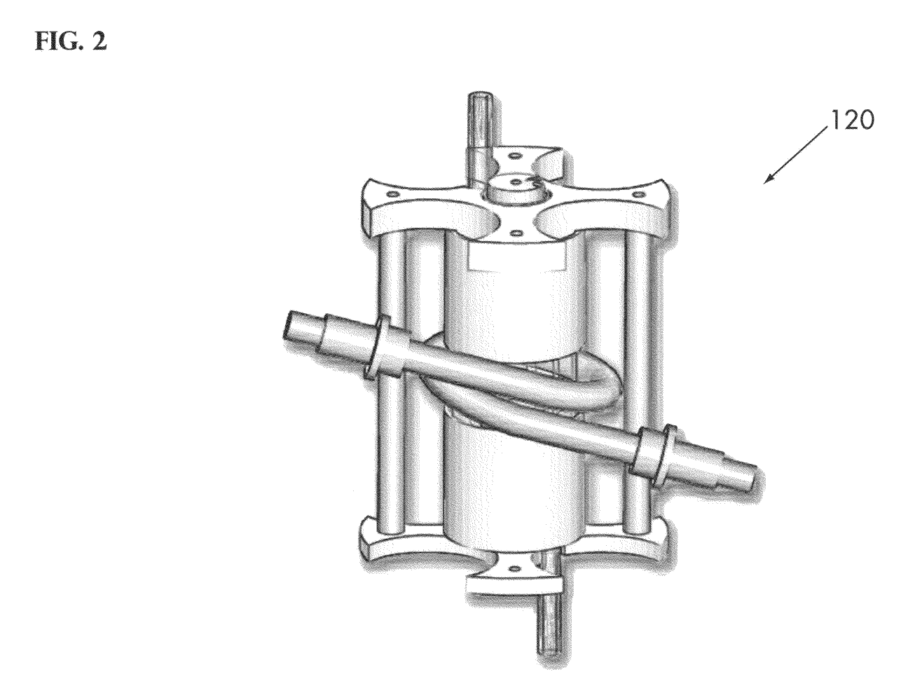

FIG. 1 illustrates a sterilization system 100 configured to provide irradiation and/or sterilization to various liquids (e.g., blood), according to some embodiments of the present invention. The following description of the system and its components will refer to a process of sterilization of blood using the system 100; however, as can be understood by one skilled in the art, the systems, devices, and methods of the present invention are applicable to any type of treatment of liquids and/or their various components.

System 100 includes a container 110 for holding an untreated liquid, a sterilization device 120, and a collection container 130 for holding a treated or sterilized liquid. The container 110 is configured to be connected to the device 120 via a supply tube 140. The container 130 is configured to be connected to the device 120 via an outlet tube 150. A liquid (e.g., blood) is supplied from the container 110 via the supply tube 140 to the device 120 for treatment (e.g., sterilization by means of application of UV radiation), and a treated liquid is collected via the outlet tube 150 inside the collection container 130.

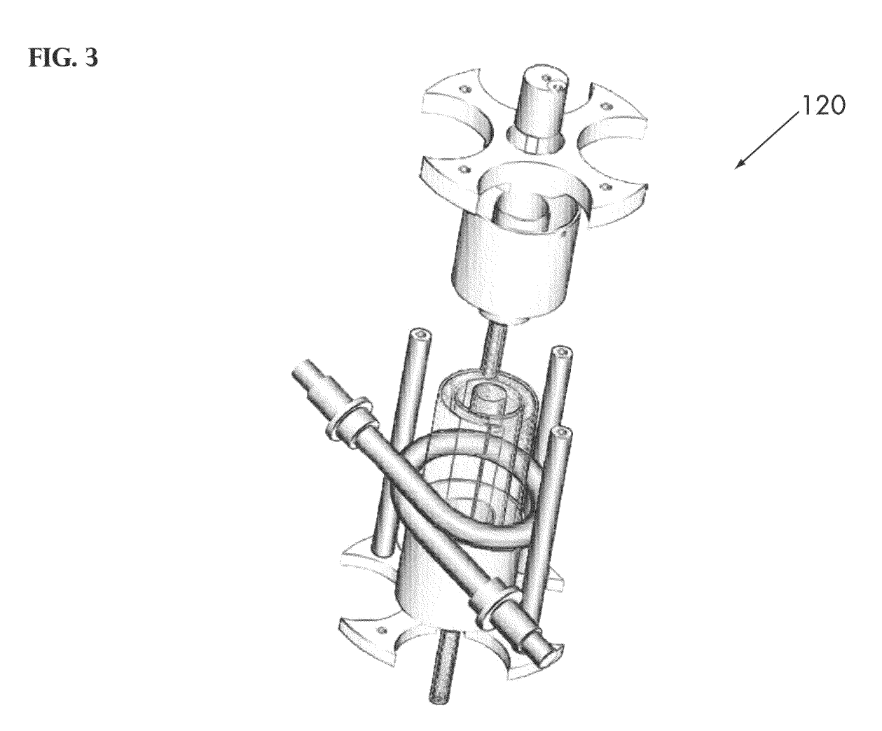

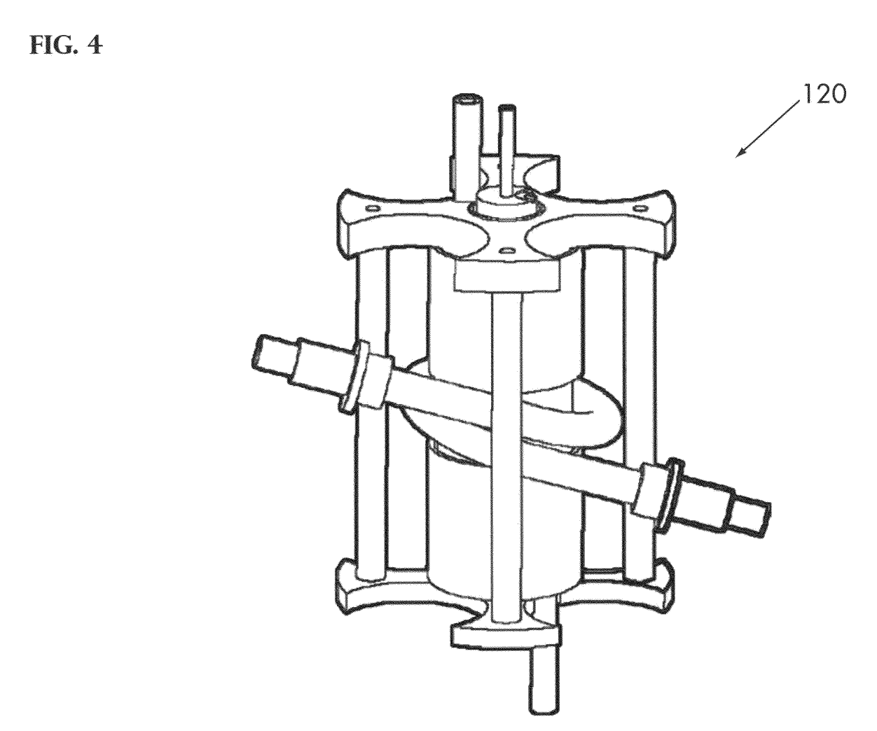

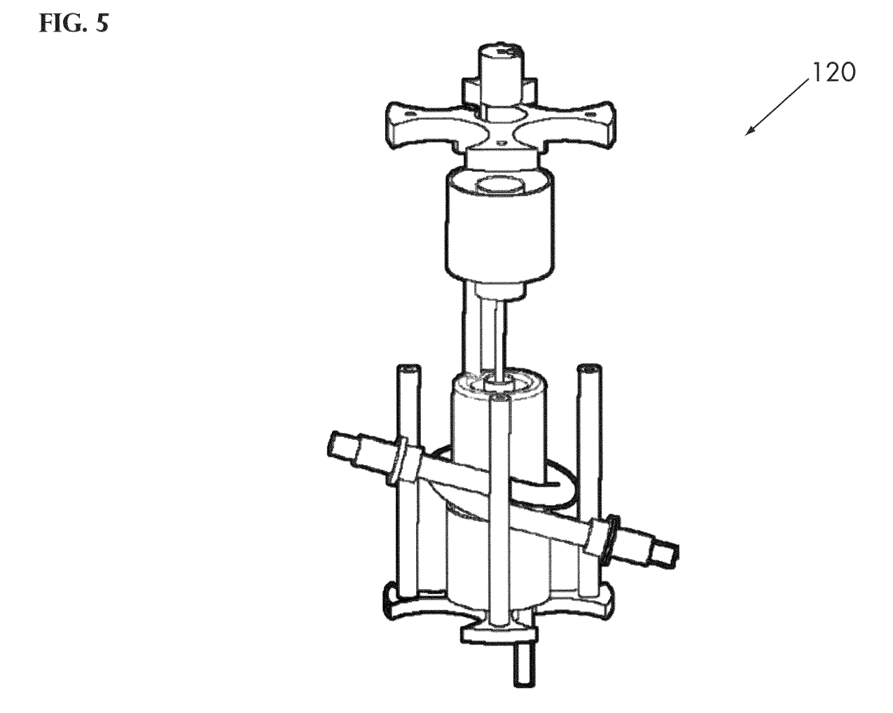

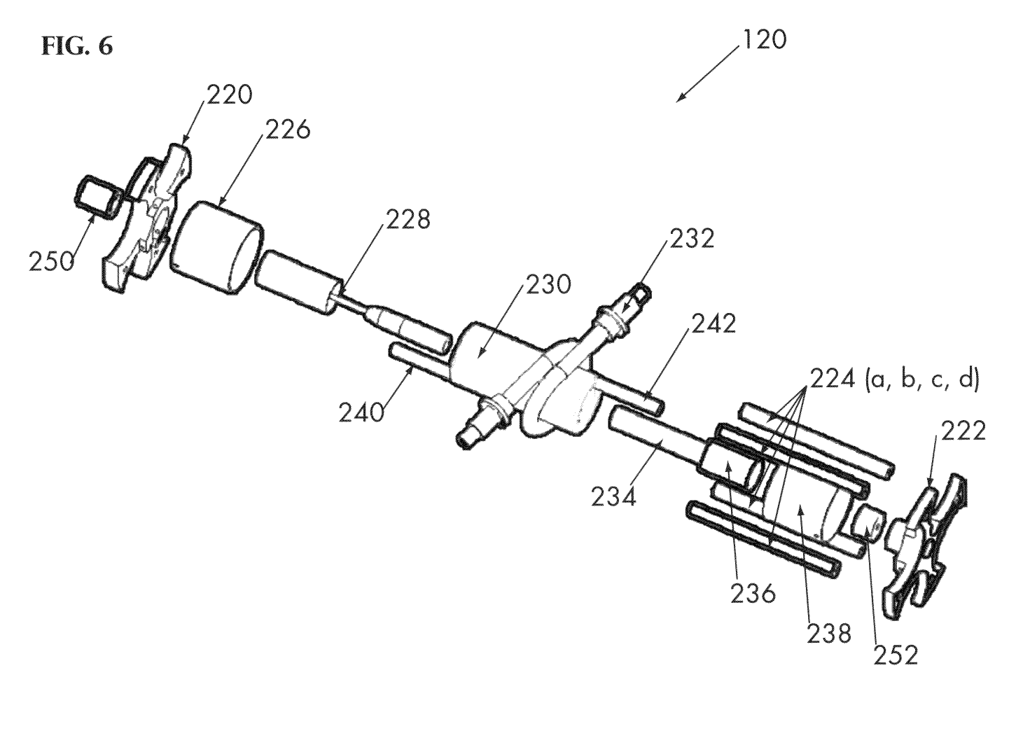

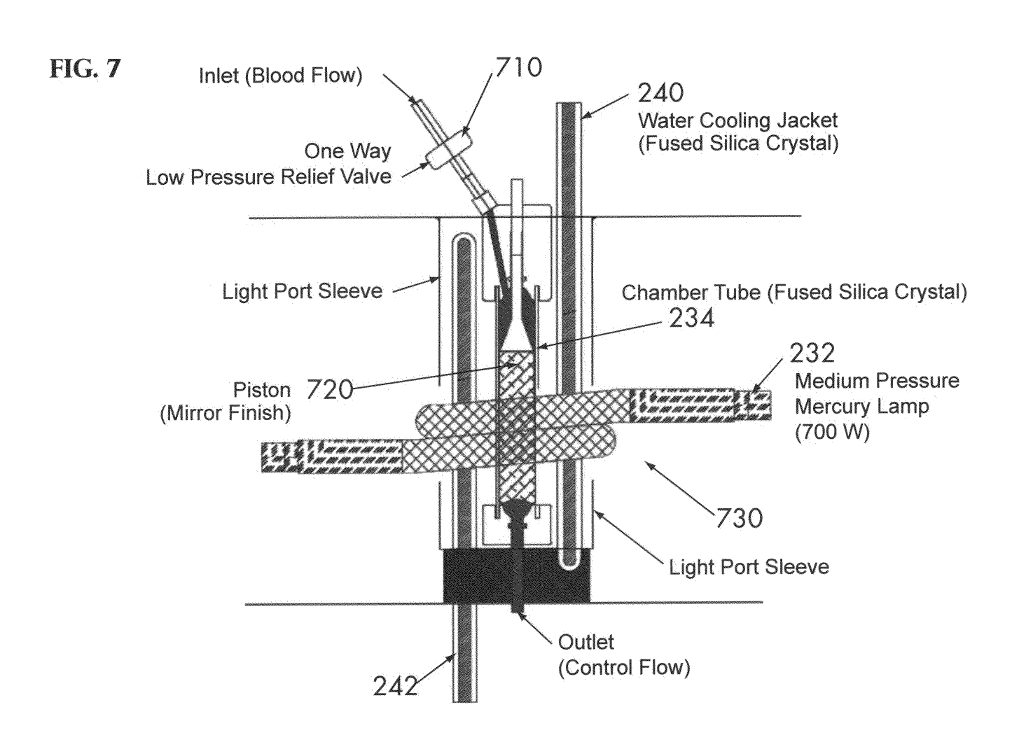

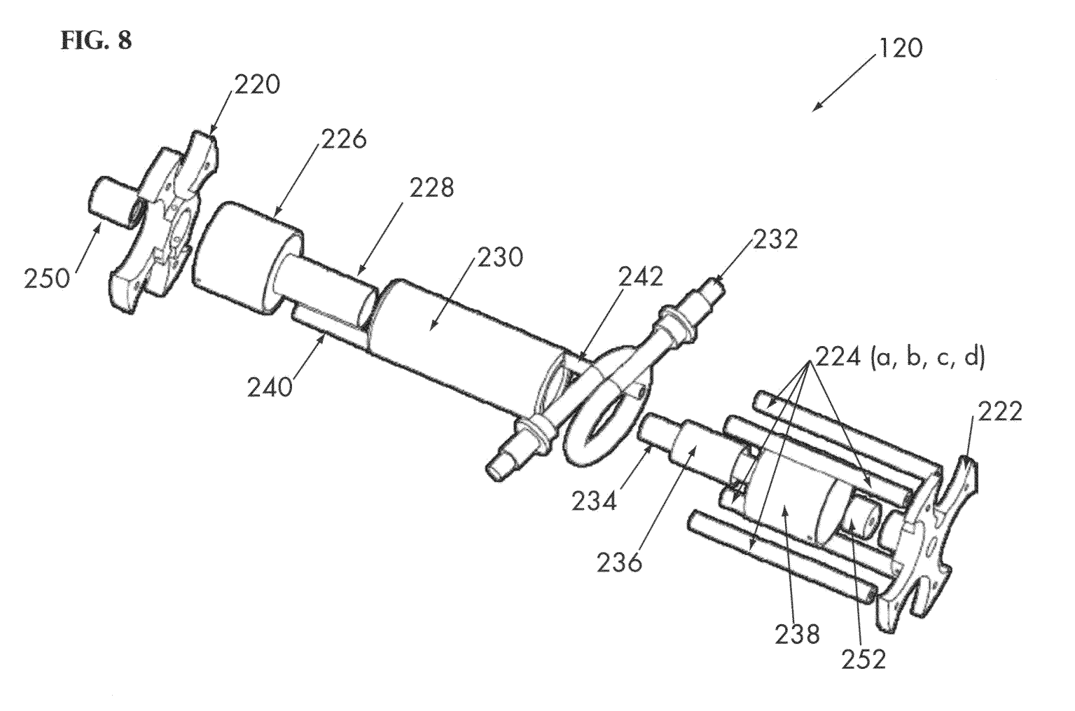

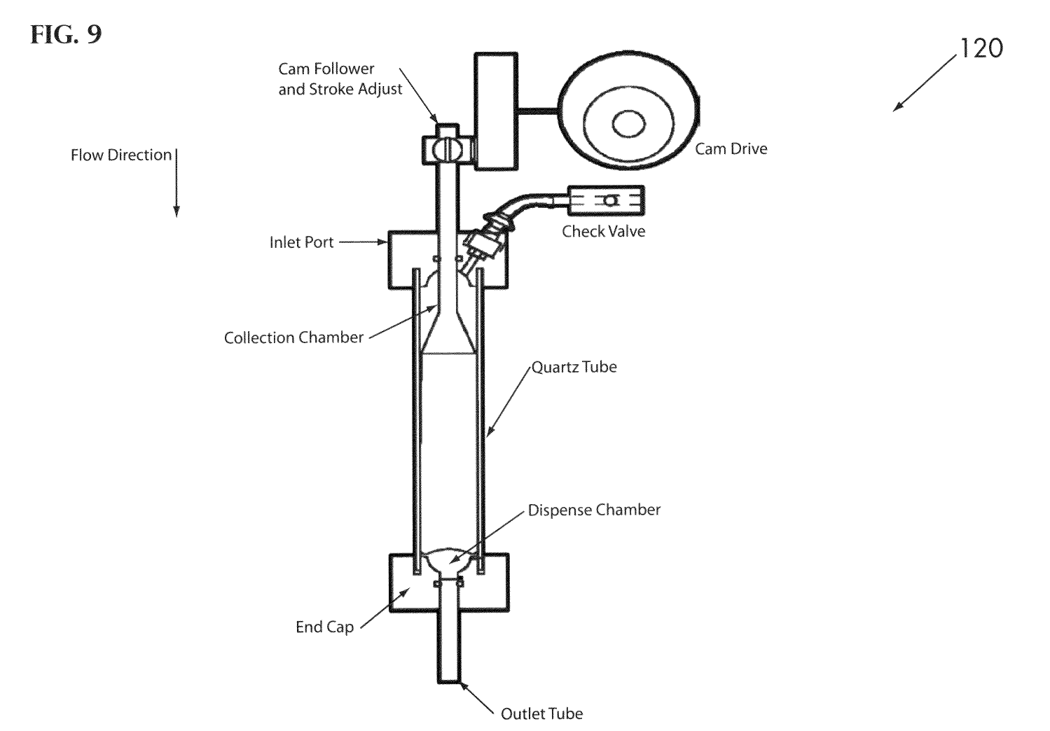

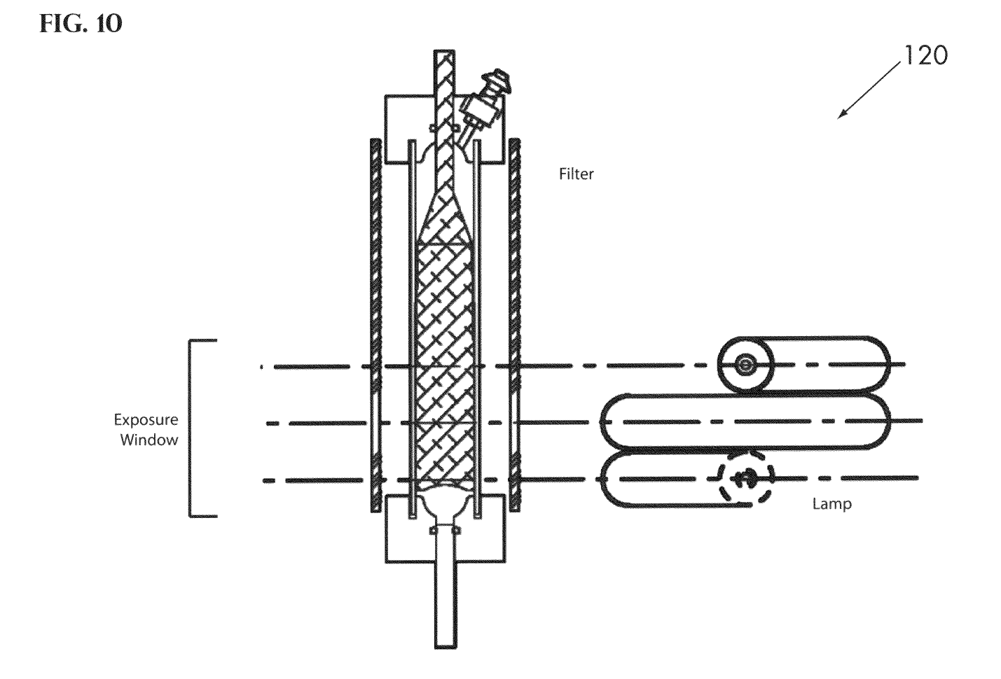

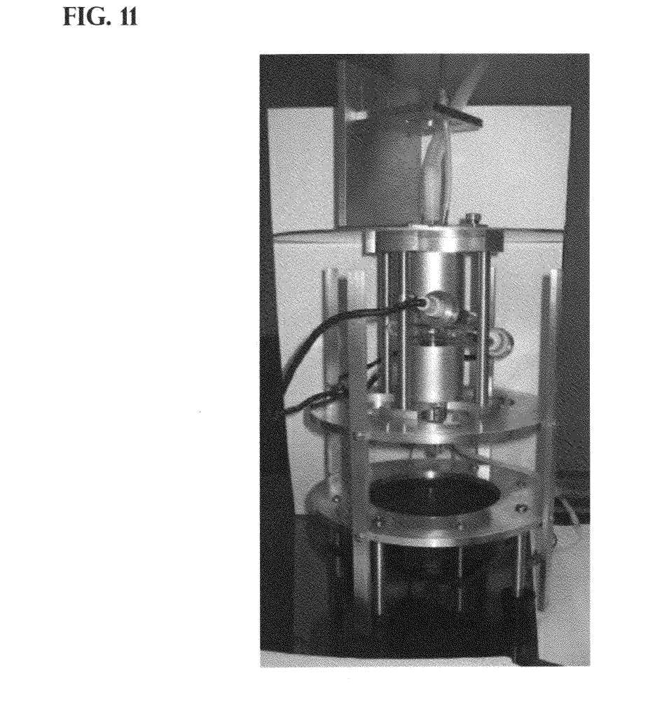

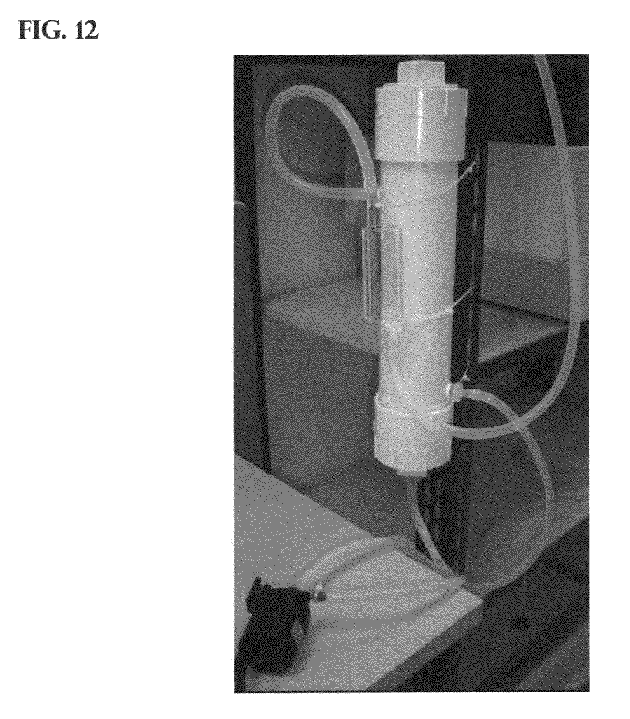

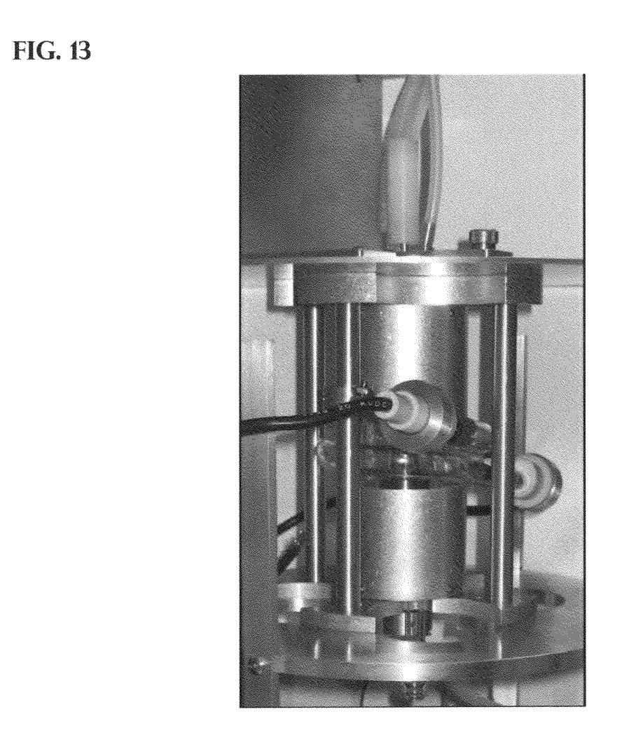

FIGS. 2-13 illustrate the sterilization device 120 and its various components in further detail. The device 120 includes lower and upper housing portions 220 and 222 coupled to each other via support posts 224 (a, b). As shown in FIG. 8, the housing portions 220 and 222 can be connected to each other via a plurality of support posts 224. The support posts 224 are configured to be disposed within four corners of the housing portions 220 and 222, as shown in FIGS. 2-6 and 8. The device 120 further includes an inlet portion 250 coupled to an inlet port assembly (226, 228), which is in turn coupled to a filter assembly 230. The filter assembly 230 is configured to include two cooling mechanisms 240 and 242 configured to be disposed along the sides of the filter assembly 230. In some embodiments, the filter assembly 230 is configured to have a cylindrical housing. As can be understood by one skilled in the art, the assembly 230 can include a housing having any other shape. The filter assembly 230 is configured to accommodate placement of a chamber assembly 234. The chamber assembly 234 is further coupled to an outlet assembly (236, 238). The outlet assembly (236, 238) is configured to be coupled to an outlet cap 252. The device further includes a lamp 232. The lamp 232 is configured to have a twisted circular shape thereby providing an all-around (360-degree) radiation of the blood flowing through the chamber 234. As can be understood by one skilled in the art, the lamp 232 can be configured to have a different shape (e.g., ellipsoidal, square, rectangular, polygonal, etc.). In some embodiments, the lamp can be configured to be a 700 W UV lamp. As can be understood by one skilled in the art, lamps having other types of power can be used. As shown in FIGS. 1, 2, and 7, in an assembled state of the device 120, the lamp 232 is configured to be disposed around the chamber 234, thereby providing the all around radiation of the liquid (e.g., blood) flowing through the chamber.

During a treatment procedure, the flow of blood proceeds from the inlet portion 250 through the filter portion 230, the chamber portion 234, where the blood is irradiated, and onto the outlet portion 252, where the blood is collected. In some embodiments, blood can be collected into an outlet reservoir.

Referring to FIGS. 7 and 9-10, the chamber 234 is illustrated in further detail. The chamber 234 is further coupled to a pump 710 for pumping blood through the chamber 234. In some embodiments, the pump 710 can be configured to include a one-way low pressure release valve. The chamber 234 further includes a piston 720 that is configured to push blood inside the chamber toward a treatment window portion 730 of the chamber 234. The piston 210 can be configured to be coupled to a motor (e.g., a stepper motor) and is further configured to have a mirror finish. During a treatment procedure, the piston 720 is configured to rotate inside the chamber and, with the chamber walls, creates a constricted location with a very narrow passage. The constricted location has a passage width in general of about 1 millimeter to about 5 millimeters to permit optimal light penetration through said bio-fluid. In some preferred embodiments, the preferred passage width is about 2 to about 4 millimeters. The piston 720 thus simultaneously translates toward the treatment window portion 730 into the constricted location passage (with a tolerance on the order of 1/1000 of an inch in this drawing) thereby pushing the blood along the walls of the chamber 234, thus, causing a smearing effect and creating a thin layer of blood flowing along the walls of the chamber toward the treatment window portion 730 and into the outlet tube. The lamp 232 is configured to be disposed around the treatment window portion or exposure window 730, so that the blood that is being pushed by the piston 720 inside the chamber 234 is treated by the UV radiation generated by the lamp 232. Upon completion of the treatment, the blood is collected in an outlet tube coupled to the chamber 234. In some embodiments, the treatment process is continuous, i.e., the blood is continuously supplied by the inlet valve into the chamber and is being pushed by the piston 720 along the interior walls of the chamber 234 for treatment, and into the outlet tube. In some embodiments, the flow/treatment rate of the blood inside the chamber 234 can be approximately 1.6 L/minute. In some embodiments, the chamber 234's housing or tube (inside which piston 720 operates) can be configured to be manufactured from a fused silica crystal.

During operation of the device 120, the lamp 232 is configured to generate a substantial amount of heat. In some embodiments, the cooling mechanisms 240 and 242 are configured to reduce the amount of heat applied to the chamber 234 during the treatment procedure. The cooling mechanism 240, 242 are configured to include water cooling jackets through having water running through them. The mechanisms 240, 242 are further configured to be disposed along the housing of the chamber 234, as shown in FIG. 7. The lamp 232 is configured to “wrap around” the cooling mechanism 240, 242, as is also shown in FIG. 7. In some embodiments, the water can be supplied to the cooling jackets of the mechanisms 240, 242 using a pump and is further distilled and air-free, thereby preventing inconsistent application of heat to the chamber 234 by the lamp 232 as well as inconsistent cooling. In some embodiments, the temperature of the water running through the mechanisms 240, 242 can be on the order of 90 degrees F., thus, cooling the chamber 234 to about 96 degree F. In some embodiments, a separate fan (not shown in FIG. 7) can be used to further cool the chamber 234. In some embodiments, use of water in the cooling mechanism 240, 242 is advantageous as it reduces application of near IR radiation to the chamber 234 in areas outside the treatment window portion 730, since water serves to absorb the near IR spectrum radiation.

In some embodiments, the housing of the chamber 234 (i.e., the fused silica crystal) can be configured to be slightly positively charged, whereas the surface of the piston 720 can be configured to be slightly negatively charged. This allows for a more effective treatment of blood, as some components (e.g., proteins, pathogens) present in the blood and to be eliminated from it are negatively charged.

Example embodiments of the methods and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

What is claimed:

1. A treatment device for treatment of a bio-fluid, comprising:
a treatment chamber configured to receive bio-fluid to be treated;
a light treatment element disposed around at least a portion of the treatment chamber;
a piston mechanism disposed inside the treatment chamber for advancing the bio-fluid inside the treatment chamber toward a constricted location corresponding to the at least a portion of the treatment chamber around which the treatment element is being disposed, said constricted location having a passage width of about 1 millimeter to about 5 millimeters to permit optimal light penetration through said bio-fluid;
wherein upon passing of the bio-fluid through the location, the treatment element applies a light treatment to the passing bio-fluid.

2. The device according to claim 1, wherein the bio-fluid is selected from the group consisting of blood and blood products.

3. The device according to claim 1, wherein the light treatment element is an ultraviolet (“UV”) lamp and the treatment is UV radiation.

4. The device according to claim 1, further comprising cooling mechanisms disposed along the treatment chamber and wherein the treatment element is configured to be disposed around the cooling mechanism and the treatment chamber.

5. The device according to claim 4, wherein each said cooling mechanism includes a cooling jacket containing water.

6. The device according to claim 5, wherein said water is distilled and air-free.

7. The device according to claim 4, wherein said cooling mechanisms are configured to reduce temperature of bio-fluid inside said treatment chamber during said treatment.

8. The device according to claim 1, wherein the piston mechanism is configured to be coupled to a motor;
said motor is configured to simultaneously translate and rotate and vertically pulse said piston mechanism inside said treatment chamber during said treatment, thereby causing the bio-fluid to spread along interior walls of said treatment chamber with shear force application.

9. The device according to claim 8, wherein said treatment chamber is further coupled to an untreated bio-fluid container for supplying bio-fluid to the treatment chamber using a pumping mechanism.

10. The device according to claim 9, wherein upon receiving bio-fluid from said untreated bio-fluid container, said piston mechanism is configured to advance said received bio-fluid inside said treatment chamber for treatment.

11. The device according to claim 10, wherein upon treating the bio-fluid inside said treatment chamber using said treatment element, said treated bio-fluid is collected inside a collection container coupled to said treatment chamber.

12. The device according to claim 1, wherein said constricted passage width is about 2 millimeters to about 4 millimeters.

13. The device according to claim 1 wherein said constricted passage includes charged inside surfaces adapted to attract oppositely charged pathogens to said inside surfaces to increase pathogen exposure to said light treatment.

14. A method for treating a bio-fluid using a treatment device for treatment of a bio-fluid, wherein the treatment device includes:
a treatment chamber configured to receive bio-fluid to be treated;
a light treatment element disposed around at least a portion of the treatment chamber;
a piston mechanism disposed inside the treatment chamber for advancing the bio-fluid inside the treatment chamber toward a constricted location corresponding to the at least a portion of the treatment chamber around which the treatment element is being disposed, said constricted location having a passage width of about 1 millimeter to about 5 millimeters to permit optimal light penetration through said bio-fluid;
wherein upon passing of the bio-fluid through the location, the treatment element applies a treatment to the passing bio-fluid;
the method comprising the steps of:
receiving bio-fluid to be treated;
using the piston mechanism, advancing the bio-fluid inside the treatment chamber;
treating the bio-fluid inside the treatment chamber; and,
collecting the treated bio-fluid in a collection container coupled to the treatment chamber.

15. The method according to claim 14, wherein the bio-fluid is selected from the group consisting of blood and blood products.

16. The method according to claim 14, wherein the treatment element is an ultraviolet (“UV”) lamp and the treatment is UV radiation.

17. The method according to claim 14, further comprising cooling the bio-fluid inside the treatment chamber during said treating step.

18. The method according to claim 14, wherein the treatment device piston mechanism is configured to be coupled to a motor; and,
said motor is configured to simultaneously translate and rotate and vertically pulse said piston mechanism inside said treatment chamber during said treatment, thereby causing the bio-fluid to spread along interior walls of said treatment chamber with shear force application.

19. The method according to claim 14, wherein said treatment device constricted passage width is about 2 millimeters to about 4 millimeters.

20. The method according to claim 14 wherein said treatment device constricted passage includes charged inside surfaces adapted to attract oppositely charged pathogens to said inside surfaces to increase pathogen exposure to said light treatment.
